(19) United States
(12) Patent Application Publication
(10) Pub. No.: US 2004/0229951 A1
(43) Pub. Date: Nov. 18, 2004

(54) Method of treating fatigue by enhancing the effectiveness of the human immune system

(76) Inventor: Arthur Vanmoor, Boca Raton, FL (US)

Correspondence Address: OTTO S. KAUDER, 5382C Venetia Ct., Boynton Beach, FL, 33437, US

(21) Appl. No.: 10/439,207

(22) Filed: May 15, 2003

(51) Int. Cl. A61K 31/205; A61K 31/198

(52) U.S. Cl. 514/563; 514/564; 514/554

(57) ABSTRACT

There is disclosed a method of treating fatigue and related ill effects of circulatory disturbance in a person in need of such treatment, which comprises the administration to such person of an effective amount of at least one compound having the formula (I)

X—CH₂—CH(OY)_{q}(H)_{1-q}CH(NH₂⁺)_{1-q}COO⁻ (I)

wherein X represents a trimethylammonio group (CH3)3N+ or a carbamoyl group (CONH₂), Y is hydrogen or an acetyl (CH₃CO) group, and q is zero or one, provided that when X is a carbamoyl group q is zero, and when X is a trimethylammonio group. q is one.

### BACKGROUND OF THE INVENTION

1. Field of the Invention

This invention relates to a method of treating a person suffering from the ill effects of circulatory disturbance including fatigue, chest pains, and elevated blood pressure with an agent that enhances the effectiveness of the human immune system to mitigate and where possible eliminate the effects of circulatory disturbance.

2. Description of Related Art

The human immune system functions to maintain human individuality by fighting off foreign entities. The MERCK MANUAL, 16^{th} edition, published 1992, at pages 279 to 303, which portion is here incorporated by reference, contains a detailed description of the parts of the immune system and of immunodeficiency diseases and hypersensitivity disorders to which it is subject. A table at pages 284-5 titled “Cytokines” lists the major effects of such cytokines or immunoeffective polypeptides as interleukin types, interferon types, alpha- and beta-tumor necrosis factor, three types of colony-stimulating factor, and alpha- and beta-transforming growth factor. A table at page 303 lists disorders with increased susceptibility to unusual infections.

The same reference at pages 400-405 provides the normal values of various parameters associated with the heart and the circulatory system and information about how various circulatory disturbances affect these parameters, including pressures within the heart and the large vessels, levels of blood oxygen and carbon dioxide, and measures of cardiac output.

Nearly everyone feels tired following physical labor or vigorous exercise such as running, jogging, climbing and calisthenics. The phenomenon is known as fatigue and can be accompanied by pain in the chest and in the particular muscle groups utilized. As the muscles function, they derive the required energy from successive chemical reactions in which nutrient substances from the blood stream are converted ultimately to carbon dioxide and water with release of energy. However, these reactions are not always 100% efficient and the gradual accumulation of incompletely metabolized by-products is believed to be responsible for the discomfort and pain known as fatigue.

While rest has been the traditional remedy for fatigue, more specific and rapidly acting remedies for fatigue and related circulatory disturbances have been sought by a great variety of methods. However, the search by scientific techniques for better remedies for this as well as other suffering conditions is enormously costly. For economic reasons, moreover, the search tends to be skewed in the direction of finding novel remedies proprietary to their discoverers and owners. Novel remedies, of course, come into being with nothing known about either their safety or their effectiveness, so that both of these essential attributes need to be exhaustively studied before they can be used as intended.

In contrast, the art has tended to neglect the exploration of therapeutic properties of known substances that humans have been safely ingesting for untold generations. Along these lines, the present inventor has been able to bring about in susceptible individuals within a limited and reproducible time the appearance of headache, elevated blood pressure, facial pimples, signs of the so-called common cold, and pains in a joint by administering selected foods, food ingredients, and relatively harmless household chemicals as trigger substances, and to use these as research tools to study the effectiveness of certain nutrient substances in relieving these artificially produced conditions as well as their natural counterparts. As a result, certain water soluble amino carboxylic acid compounds are disclosed in U.S. Pat. No. 5,616,617 as effective against facial pimples; certain water soluble amino carboxylic acid compounds are disclosed in U.S. Pat. No. 5,626,831 as effective against the common cold; certain water soluble amino carboxylic acid compounds are disclosed in U.S. Pat. No. 5,707,967 as effective against headache; certain water soluble amino carboxylic acid compounds are disclosed in U.S. Pat. No. 5,708,029 as effective against elevated blood pressure, and certain water soluble amino carboxylic acid compounds are disclosed in U.S. Pat. No. 5,767,157 as effective against pain in a joint.

Published PCT application PCT/US 01/00714 discloses treatment of an ailment, which can be fatigue and circulatory disturbance, with an aliphatic sulfur compound.

### SUMMARY OF THE INVENTION

In accordance with this invention, there is provided a method of treating fatigue and related ill effects of circulatory disturbance in a person in need of such treatment, which comprises the administration to such person of an effective amount of at least one compound represented by formula (I) shown below.

X—CH₂—CH(OY)_{q}(H)_{1-q}CH(NH₂⁺)_{1-q}COO³¹ (I)

wherein X represents a trimethylammonio group (CH3)3N+ or a carbamoyl group (CONH₂), Y is hydrogen or an acetyl (CH₃CO) group, and q is zero or one, provided that when X is a carbamoyl group q is zero, and when X is a trimethylammonio group q is one.

Accordingly, in the compound represented by formula (I), when X represents a trimethylammonio group (CH3)3N+ the compound is represented by formula (II)

(CH3)3N+CH₂—CH(OY)CH₂COO⁻ (II)

wherein Y is hydrogen or an acetyl (CH₃CO) group as defined above.

In the compound represented by formula (I), when X represents a carbamoyl group (CONH₂) the compound is represented by formula (III)

(CONH₂)CH₂—CH₂CH(NH₂⁺)COO⁻ (III)

The effectiveness of the compound represented by formula (I) according to the invention is believed to be distinct from the effect of such compound as a nutrient, and is believed to accompany enhancement of the effectiveness of the person's immune system.

The present invention is based on the recognition that enhancing the effectiveness of the immune system in a person can be beneficial in augmenting the person's innate ability to resist and recover from fatigue associated with physical activity including the uncomfortable and potentially dangerous effects of circulatory disturbance. Without intending to be bound by any theory, it is believed that by way of the immune system the accumulation of metabolic byproducts in muscles is slowed or the further conversion of such by-products is accelerated. Thus, fatigue can be diminished when it is perceived by administration of a compound represented by formula (I) according to this invention. However, perception of fatigue can also be postponed or avoided altogether by administration of at least one compound represented by formula (I) according to this invention when the possible occurrence of fatigue is anticipated. As a result, the quality of life is improved.

In increasing the effectiveness of the human immune system according to this invention, doses of 2 to 20 grams of a compound or compounds of formula (I) can be administered to a person after signs of fatigue or other circulatory disturbance appear from one to five times a day for a total of 2 to 100 grams per day, preferably 2 to 50 grams per day,. in order to diminish the intensity and duration of fatigue Such doses can also be administered in advance of or simultaneously with incidence of physical stress. Doses can be administered in any convenient manner, as by oral administration in any of the usual dosage forms, such as tablets, capsules, solutions, and dispersions in liquid foods such as soups and fruit juices. Alternatively, there can be given sterile solutions by direct injection into the bloodstream of the person to be treated, as well as by rectal suppositories. When improvement is noted, the dose can be reduced to a maintenance level of about 10 grams daily.

### EXAMPLE 1

A 48 year old man who experienced heaviness and tingling in both legs found complete relief within a few hours after taking the first of four ten gram doses in one day of a mixture of compound represented by formula (II) and compound represented by formula (III)

### EXAMPLE 2

A 58 year old woman regularly felt chest pains after once a day running 20 minutes on a horizontal belt machine at 4.5 miles per hour. She took a single dose of 25 grams of compound represented by formula (I) ten minutes before starting to run the next day and was free of pain running for 30 minutes at the same speed.

### EXAMPLE 3

Volunteers afflicted by fatigue in their legs took 40 to 50 grams daily of composition containing compounds of formula I and overcame the fatigue. They also noted lower blood pressure, longer endurance in running, and reduced incidence of chest pains.

What is claimed is:

1. A method of treating effects of fatigue in a person in need of such treatment, which comprises the administration to such person of an effective amount of at least one compound represented by formula (I)
X—CH₂—CH(OY)_{q}(H)_{1-q}CH(NH₂⁺)_{1-q}COO⁻ (I)
wherein X represents a trimethylammonio group (CH3)3N+ or a carbamoyl group (CONH₂), Y is hydrogen or an acetyl (CH₃CO) group, and p and q is zero or one, provided that when X is a carbamoyl group, q is zero, and when X is a trimethylammonio group. q is one.

2. The method of claim 1 wherein the compound is represented by formula (II)
(CH3)3N+CH₂—CH(OY)CH₂COO⁻ (II)
wherein Y is hydrogen or an acetyl (CH₃CO) group

3. The method of claim 1 wherein the compound is represented by formula (III)
(CONH₂)CH₂—CH₂CH(NH₂⁺)COO⁻ (III)

4. The method of claim 1 wherein the compound is administered orally when fatigue is observed.

5. The method of claim 1 wherein the compound is administered orally prior to or simultaneously with occurrence of fatigue.

6. The method of claim 1, wherein the compound is administered by injection into the bloodstream.

7. The method of claim 1, wherein the compound is administered by rectal suppository.

8. The method of claim 1, wherein the compound is administered in one to five daily doses of 2 to 20 grams each.

9. The method of claim 1, wherein the total of the compound administered daily is in the range of 2 to 100 grams.

10. The method of claim 9, wherein the total of the compound administered daily is in range of 2 to 50 grams
